(19) United States
(12) Patent Application Publication
(10) Pub. No.: US 2026/0345943 A1
(43) Pub. Date: Nov. 5, 2026

(54) POWDER DELIVERY DEVICES AND METHODS OF USE

(71) Applicant: Boston Scientific Scimed, Inc., Maple Grove, MN (US)

(72) Inventors: John Joseph SUBASIC, Sutton, MA (US); Aaron Robert ROSENTHAL, Foxboro, MA (US); Lauren Sfakis LYDECKER, Upton, MA (US)

(73) Assignee: Boston Scientific Scimed, Inc., Maple Grove, MN (US)

(21) Appl. No.: 19/661,928

(22) Filed: Apr. 29, 2026

Related U.S. Application Data
(60) Provisional application No. 63/798,041, filed on May 1, 2025.

(51) Int. Cl. A61M 16/14; A61M 15/00; A61M 16/20

(52) U.S. Cl. CPC A61M 16/14; A61M 15/0008; A61M 16/201; A61M 2202/064

(57) ABSTRACT

A device for delivering an agent that includes an inlet configured to receive a pressurized fluid and an enclosure configured to store the agent. The device includes an actuator positioned between and in fluid communication with each of the inlet and the enclosure, the actuator including an opening. The actuator is configured to deliver the pressurized fluid from the inlet towards the enclosure in response to the opening being sealed, thereby inhibiting the pressurized fluid from exiting the device via the opening. The actuator is configured to inhibit delivery of the pressurized fluid from the inlet towards the enclosure in response to the opening being unsealed, thereby allowing the pressurized fluid to exit the device via the opening.

### CROSS-REFERENCE TO RELATED APPLICATIONS

This application claims the benefit of priority to U.S. Provisional Application No. 63/798,041, filed on May 1, 2025, which is incorporated by reference herein in its entirety.

### TECHNICAL FIELD

Various embodiments of the present disclosure relate generally to devices and methods for delivering agents. More specifically, this disclosure relates to devices for delivery of powdered agents, such as prophylactic agents.

### BACKGROUND

In certain medical procedures, it may be necessary to minimize or stop bleeding internal to the body. For example, an endoscopic medical procedure may require hemostasis of bleeding tissue within the gastrointestinal tract, for example in the esophagus, stomach, or intestines. During an endoscopic procedure, a user inserts a sheath of an endoscope into a body lumen of a patient. The user utilizes a handle of the endoscope to control the endoscope during the procedure. Tools may be passed through a working channel of the endoscope via, for example, a port in the handle, to deliver treatment at the procedure site near a distal end of the endoscope. The procedure site is remote from the user.

To achieve hemostasis at the remote site, an agent (e.g., a prophylactic bio-adhesive) may be delivered by a delivery device inserted into the working channel of the endoscope. Agent delivery may be achieved, for example, through mechanical systems. Such systems, however, may require numerous steps or actuations to achieve delivery, may not achieve a desired rate of agent delivery or a desired dosage of agent, may result in the agent clogging portions of the delivery device, may result in inconsistent dosing of the agent, and/or may not result in the agent reaching the treatment site deep within the gastrointestinal tract. The current disclosure may solve one or more of these issues or other issues in the art.

### SUMMARY OF THE DISCLOSURE

Aspects of the disclosure relate to, among other things, systems, devices, and methods for a medical device including a valve assembly for delivering an agent (e.g., a prophylactic powder, a hemostatic powder, a bio-adhesive, etc.). More specifically, this disclosure relates to devices for controlled delivery of powdered agents, such as prophylactic agents, via a valve assembly that includes an enclosure for storing the prophylactic agent and one or more valves that are movably disposed inside the device to facilitate selective delivery of a pressurized fluid and the prophylactic agent out of the device. Each of the aspects disclosed herein may include one or more of the features described in connection with any of the other disclosed aspects.

According to an example, a device for delivering an agent comprises an inlet configured to receive a pressurized fluid; an enclosure configured to store the agent; and an actuator positioned between and in fluid communication with each of the inlet and the enclosure, the actuator including an opening; wherein the actuator is configured to deliver the pressurized fluid from the inlet towards the enclosure in response to the opening being sealed, thereby inhibiting the pressurized fluid from exiting the device via the opening; and wherein the actuator is configured to inhibit delivery of the pressurized fluid from the inlet towards the enclosure in response to the opening being unsealed, thereby allowing the pressurized fluid to exit the device via the opening.

Any of the devices described herein may include any of the following features. A handle including the inlet and the actuator, wherein the actuator includes a button formed along the handle such that the opening is disposed along an exterior of the handle. The button is configured to receive a finger of a user to selectively seal and unseal the opening for delivering the pressurized fluid towards and away from the enclosure. A bypass channel positioned between the actuator and the enclosure, the bypass channel being in fluid communication with the inlet via the actuator for receiving the pressurized fluid. The bypass channel is configured to receive a portion of the pressurized fluid from the actuator in response to the opening being sealed; and wherein the bypass channel extends around the enclosure such that the bypass channel is configured to deliver the portion of the pressurized fluid received from the actuator to an outlet of the device that is positioned downstream from the enclosure. A first valve disposed downstream from the bypass channel, wherein the first valve is configured to selectively direct the portion of the pressurized fluid towards the outlet based on a position of the first valve. When in a first position, the first valve is configured to close the bypass channel to inhibit the portion of the pressurized fluid from moving towards the outlet and exiting the device; and wherein when in a second position, the first valve is configured to open the bypass channel to allow the portion of the pressurized fluid to move towards the outlet and exit the device. A second valve disposed downstream from the enclosure, wherein the second valve is configured to selectively direct the agent stored in the enclosure towards the outlet based on a position of the second valve. When in a first position, the second valve is configured to close an opening of the enclosure to inhibit the agent from moving towards the outlet and exiting the device; and wherein when in a second position, the second valve is configured to open the opening of the enclosure to allow the agent to move towards the outlet and exit the device. The enclosure includes an interior surface defining a cavity that is configured to store the agent, and an agitation mechanism extending from the interior surface into the cavity; and wherein the agitation mechanism is configured to agitate the pressurized fluid received in the enclosure from the actuator and the agent stored in the cavity, thereby forming a mixture of the pressurized fluid and the agent within the enclosure for delivery. The agitation mechanism includes a plurality of ribs configured to abut against the agent in response to the pressurized fluid received in the enclosure moving the agent within the cavity. The agitation mechanism includes a rotatable disc that is configured to rotate within the enclosure and generate a vortex within the cavity in response to the pressurized fluid received in the enclosure moving the rotatable disc. The actuator includes a first actuator and the opening includes a first opening; wherein the device further comprises: an outlet; and a second actuator positioned between and in fluid communication with each of the inlet and the outlet, wherein the second actuator includes a second opening. The second actuator is configured to deliver the pressurized fluid from the inlet towards the outlet in response to the second opening being sealed, thereby inhibiting the pressurized fluid from exiting the device via the second opening; and wherein the second actuator is configured to inhibit delivery of the pressurized fluid from the inlet towards the outlet in response to the second opening being unsealed, thereby allowing the pressurized fluid to exit the device via the second opening. The first actuator is configured to selectively deliver a first portion of the pressurized fluid received from the inlet towards the enclosure and the second actuator is configured to selectively deliver a second portion of the pressurized fluid received from the inlet towards the outlet independently from one another based on each of the first opening and the second opening being sealed or unsealed.

According to another example, a device for delivering an agent, comprising: an enclosure configured to store the agent; an inlet configured to receive a pressurized fluid; an outlet configured to deliver the agent and the pressurized fluid out of the device; a first actuator positioned between and fluidly coupled to the inlet and the enclosure, wherein the first actuator is configured to selectively guide the pressurized fluid from the inlet towards the enclosure for mixing with the agent when the first actuator is covered, and release the pressurized fluid from the inlet out of the device when the first actuator is uncovered; and a second actuator positioned between and fluidly coupled to the inlet and the outlet, wherein the second actuator is configured to selectively guide the pressurized fluid from the inlet towards the outlet for when the second actuator is covered, and release the pressurized fluid from the inlet out of the device when the second actuator is uncovered.

Any of the devices described herein may include any of the following features. The first actuator and the second actuator are configured to simultaneously guide the pressurized fluid into the enclosure for mixing the agent and towards the outlet for delivering the agent released from the enclosure towards the outlet when each of the first actuator and the second actuator are covered. The first actuator and the second actuator are configured to guide the pressurized fluid away from the enclosure and towards the outlet for delivering the pressurized fluid towards the outlet in isolation from the agent when the first actuator is uncovered and the second actuator is covered. The first actuator and the second actuator are configured to guide the pressurized fluid away from the enclosure and the outlet, thereby releasing the pressurized fluid prior to mixing with the agent and entering the outlet, when each of the first actuator and the second actuator are uncovered.

According to a further example, a system for delivering an agent, comprising: a first device including a handle and a delivery conduit that is in fluid communication with the handle; and a second device including an actuator, an inlet configured to receive a pressurized fluid, and an enclosure configured to store the agent; wherein the actuator is configured to fluidly couple the first device and the second device to selectively deliver the agent from the enclosure to the delivery conduit in response to the actuator moving from an unsealed state to a sealed state; wherein the actuator is configured to divert the pressurized fluid received from the inlet away from the enclosure and out of the second device prior to the pressurized fluid entering the first device when in the unsealed state; and wherein the actuator is configured to urge the pressurized fluid received from the inlet into the enclosure and out of the second device for receipt by the first device when in the sealed state.

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “exemplary” is used in the sense of “example,” rather than “ideal.” The term “approximately,” or like terms (e.g., “substantially”), includes values +/- 10 % of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 shows an exemplary delivery device, according to one or more embodiments.

FIG. 2 shows a partial side view of an exemplary valve assembly of the delivery device of FIG. 1, according to one or more embodiments.

FIG. 3 shows a partial top view of an exemplary control button of the valve assembly of FIG. 2, according to one or more embodiments.

FIG. 4 shows a partial side view of another exemplary valve assembly of the delivery device of FIG. 1, according to one or more embodiments.

FIG. 5 shows a partial side view of another exemplary valve assembly of the delivery device of FIG. 1, according to one or more embodiments.

FIG. 6 shows a partial side view of an exemplary medical system, according to one or more embodiments.

FIG. 7 shows a partial side view of another exemplary valve assembly of the delivery device of FIG. 1, according to one or more embodiments.

### DETAILED DESCRIPTION OF EMBODIMENTS

Embodiments of this disclosure relate to dispensing devices having valve assemblies for releasing a pressurized fluid (e.g., gas, air, liquid, etc.) and a material (e.g., a powdered agent) to a site of a medical procedure. In other words, the valve assemblies may be configured and operable to move stagnant material and facilitate proper dispensing of the material via the pressurized fluid. The valve assembly may include one or more valves movably disposed inside device for selectively controlling delivery of a pressurized fluid and agent stored in an enclosure, in order to combine the agent and pressurized fluid. The enclosure may store a predetermined amount of the agent, and may be in fluid communication with a pressurized fluid source storing the pressurized fluid. Upon release of the pressurized fluid into the enclosure by the one or more valves, the agent and the fluid may be combined within the enclosure prior to delivery from the device.

The valve assembly may further include one or more actuators (e.g., control buttons) for selectively controlling a flow of the pressurized fluid relative to the enclosure storing the agent, including but not limited to, directing the pressurized fluid to bypass the enclosure, towards the enclosure to mix with the agent stored therein, and/or simultaneously into the enclosure for mixing with the agent while bypassing the enclosure for isolated delivery of the pressurized fluid without the agent.

The pressurized fluid may be delivered through the valve assembly while diverting around the enclosure via the one or more valves and/or control buttons of the valve assembly to deliver the pressurized fluid in isolation without the agent. The agent stored in the enclosure may be placed in fluid communication with the pressurized fluid through actuation of at least one of the one or more valves and/or control buttons of the valve assembly. Accordingly, when the agent is combined with pressurized fluid, the valve assembly delivers the combination of the agent and the pressurized fluid through an outlet conduit to a target treatment site during a medical procedure. Aspects of the dispensing device and valve assembly, such as the one or more valves and/or control buttons, may facilitate agitation and combination of the agent and the pressurized fluid prior to dispensing to prevent or minimize clogging of the dispensing device during delivery of agent. The valve assembly may be configured and operable to move the agent that may be stored in a stagnant state within the enclosure and facilitate proper dispensing of the agent out of the dispensing device.

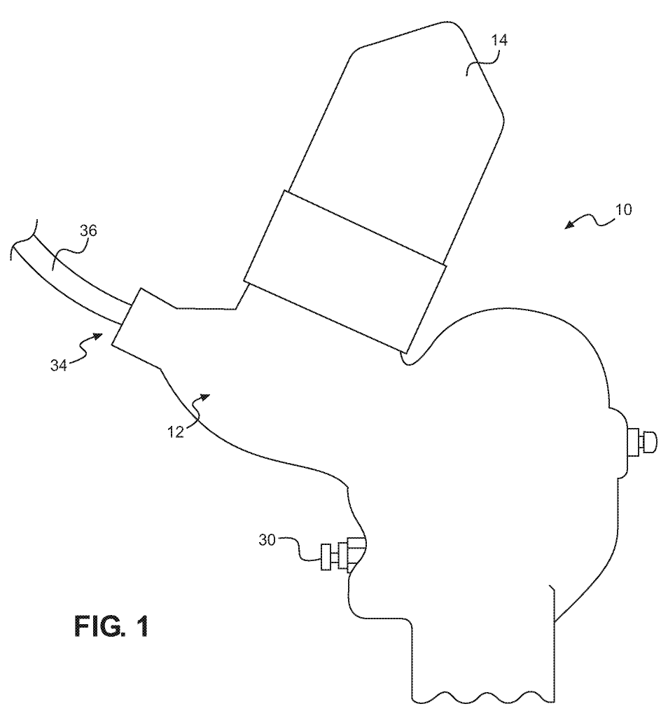

FIG. 1 shows a delivery system 10, which may be a powder delivery system. Delivery system 10 may include a handle body 12. Handle body 12 may include, or may be configured to receive, an enclosure 14 (or other source or container) storing a material (e.g., a powdered agent). Enclosure 14 may be coupled to handle body 12 for providing the agent to handle body 12, or a lid/enclosure of the agent may be screwed onto, or otherwise coupled to, enclosure 14 for supplying the agent to enclosure 14.

The agent may be, for example, a powdered agent, such as a prophylactic agent, a hemostatic agent, or other various agents. In some examples, the prophylactic agent may include a bio-adhesive operable to promote natural healing by stopping a bleeding upon application to the target treatment site. Additionally, the prophylactic agent may be operable to generate a protective covering of the target treatment site experiencing the bleeding to mitigate subsequent rebleeding occurrences. In some embodiments, the powdered agent may include a particle size of less than about 100 µm. The agent may alternatively be another type of agent or material, or form of agent (e.g., a liquid or gel agent), and may have any desired function. Enclosure 14 may be removably attached to other components of delivery system 10, including components of handle body 12.

Handle body 12 may have a variety of features, to be discussed in further detail herein. U.S. Patent Application No. 16/589,633, filed Oct. 1, 2019, published as U.S. Patent No. 11,642,281 B1 on May 9, 2023, the disclosure of which is hereby incorporated by reference in its entirety, discloses features of exemplary delivery devices and systems. The features of this disclosure may be combined with any of the features described in the above-referenced patent. The features described herein may be used alone or in combination and are not mutually exclusive. Like reference numbers and/or terminology are used to denote similar structures, when possible.

Still referring to FIG. 1, delivery system 10 may include an actuation mechanism 30 used to activate flow of a pressurized fluid (e.g., gas, air, liquid, etc.) from a pressurized fluid source (not shown) in fluid communication with delivery system 10. In some examples, the pressurized fluid source may be external to delivery system 10. In other examples, the pressurized fluid source (e.g., gas or air canister) may be disposed inside delivery system 10, such as internal to handle body 12. Actuation mechanism 30 may be selectively actuated (e.g., manually depressible) or otherwise moved or actuated to control delivery of a material (e.g., a powdered agent) and/or the pressurized fluid. The pressurized fluid alone, or a combination of a powdered agent and the pressurized fluid, may be delivered from an outlet 34 of handle body 12. Outlet 34 may be in fluid communication with a delivery conduit, for example a catheter 36 or another component for delivering the combination of agent and fluid to a desired location within a body lumen of a patient. Delivery system 10 may include a valve assembly for selectively controlling the release of the powdered agent and/or the pressurized fluid within enclosure 14.

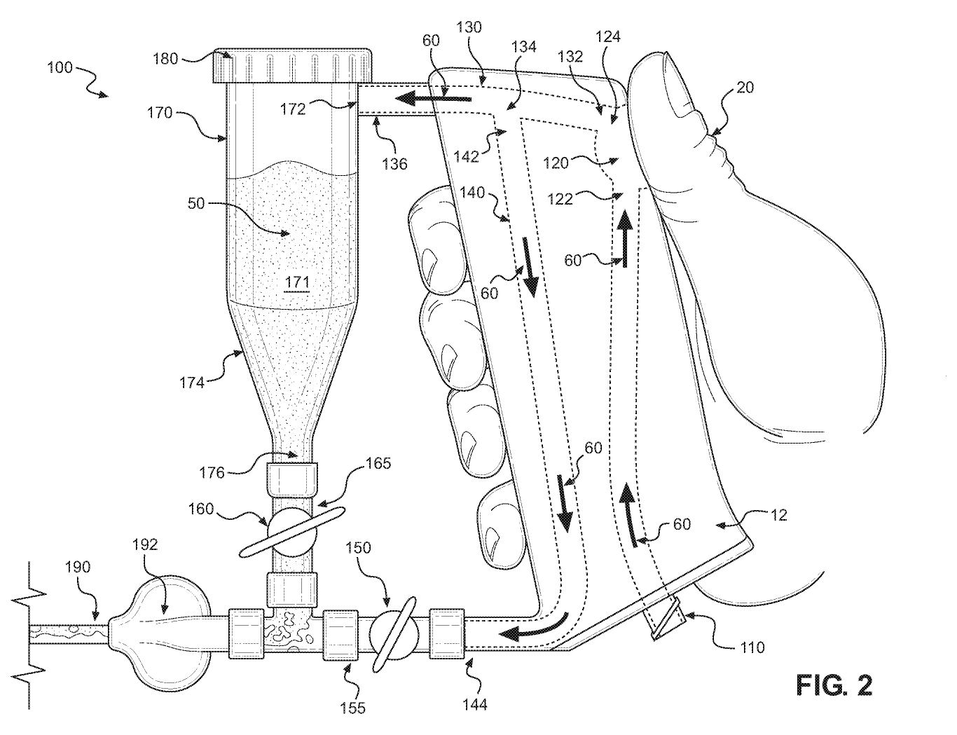

FIG. 2 shows an exemplary valve assembly 100, according to one or more embodiments. Valve assembly 100 may be part of delivery system 10 as discussed with reference to FIG. 1 above, such as fluidly coupled to enclosure 14. Valve assembly 100 may include an inlet 110 (e.g., a channel defining a lumen) in fluid communication with the pressurized fluid source (not shown) that is configured to store the pressurized fluid (e.g. gas, air, liquid, etc.), a chamber and/or enclosure 170 for storing a material, such as an agent 50 (e.g. a powdered agent), a control button 120 (e.g., an actuator) disposed along a portion of handle body 12, and a first channel 130 and a second channel 140 disposed within handle body 12. In some embodiments, the fluid source may be external to delivery system 10 (FIG. 1), such that the fluid source may be fluidly coupled to delivery system 10, and particularly valve assembly 100. Valve assembly 100 may further include a first valve 150 and a second valve 160 fluidly coupled to one or more of first channel 130, second channel 140, and/or enclosure 170. Enclosure 170 may include a cap 180 enclosing a cavity 171 of enclosure 170. Valve assembly 100 may include an outlet 190 (e.g., a tube defining a lumen) in fluid communication with a delivery conduit (e.g. catheter 36) for delivering agent 50, the pressurized fluid, and/or a combination thereof.

A predetermined amount of agent 50 may be stored within enclosure 170 for delivery from valve assembly 100. The predetermined amount of agent 50 stored therein may vary depending on an application of use, such as a treatment of a subject (e.g., patient) or a target treatment site. First valve 150 and second valve 160 may be sized, shaped, and/or otherwise configured to move, transition, and/or actuate to various different positions, states, and/or configurations as described in further detail herein. In some embodiments, one or more of valves 150, 160 may include a ball valve, a three-way stopcock valve, a luer valve, etc. In other embodiments, one or more of valves 150, 160 may be configured and operable as other suitable valving mechanisms without departing from a scope of this disclosure.

Still referring to FIG. 2, outlet 190 may be in fluid communication with inlet 110 and enclosure 170 via first valve 150 and second valve 160, respectively. Enclosure 170 may include a first opening 172 positioned adjacent at a first (upper) portion of enclosure 170, a funnel 174 disposed along an intermediate portion of enclosure 170, and a second opening 176 positioned adjacent to a second (lower) portion of enclosure 170, which may collectively define cavity 171 of enclosure 170 for storing the material, e.g., agent 50. First opening 172 may be positioned upstream from funnel 174 of enclosure 170 for fluidly connecting cavity 171 of enclosure 170 to inlet 110 via first channel 130, such that enclosure 170 may be in fluid communication with inlet 110 through first opening 172. Second opening 176 may be positioned downstream from funnel 174 of enclosure 170 for fluidly connecting enclosure 170 to outlet 190 via second valve 160, such that enclosure 170 may be in fluid communication with outlet 190 through second opening 176. Funnel 174 may be configured in a tapered, a conical, and/or an angled shape to guide agent 50 in moving within enclosure 170 towards second opening 176. In some examples, funnel 174 may be configured to direct a mixture of the pressurized fluid and agent 50 out of enclosure 170 and towards second opening 176 for delivery of the mixture of the pressurized fluid and agent 50 towards outlet 190.

Control button 120 may include an actuator that is sized, shaped, and/or otherwise configured as an opening formed on and/or positioned along handle body 12 for selective actuation by a user, and particularly a hand of the user. Stated differently, control button 120 may include an opening, a recess, a cavity, an aperture, and/or an indentation on handle body 12 that is in fluid communication with one or more internal fluid channels of valve assembly 100 (within handle body 12). In this instance, control button 120 may define a fluid valve formed as an opening on an exterior of handle body 12 that is accessible from and open to a surrounding atmosphere and/or environment. Control button 120 may be configured to at least partially receive a portion of the hand of a user, such as on, within, and/or over control button 120, or a combination thereof. In other words, control button 120 may be manually accessible by the user from an exterior of handle body 12 for manually covering the opening of control button 120 with a physical surface, such as that of a finger of the user’s hand. As described herein, control button 120 may be in fluid communication with one or more channels, inlets, and/or outlets of valve assembly 100.

Control button 120 may be configured to control a flow 60 of the pressurized fluid through valve assembly 100 via selective actuation by at least one finger 20 of the user’s hand. In the example, the at least one finger 20 may include a thumb of the user’s hand and selective actuation of control button 120 may include positioning the at least one finger 20 over control button 120. In some embodiments, control button 120 may be sized and/or shaped to include a diameter or a cross-sectional profile that is relatively smaller than the at least one finger 20. In this instance, control button 120 may be partially and/or completely sealed, concealed, covered, and/or otherwise closed off by the at least one finger 20 to actuate control button 120 during use of delivery system 10.

Still referring to FIG. 2, control button 120 may include a first opening 122 and a second opening 124 positioned along an opposing surface of control button 120 relative to first opening 122. In some embodiments, openings 122, 124 may be positioned transversely relative to one another, such that openings 122, 124 may be relatively misaligned with one another. First opening 122 may be positioned downstream from inlet 110 and configured to receive the pressurized fluid received in valve assembly 100 via inlet 110. Second opening 124 may be configured to receive the pressurized fluid passing through control button 120 from first opening 122 when finger 20 is positioned over control button 120, thereby closing and/or sealing control button 120. Second opening 124 may fluidly couple control button 120 with first channel 130.

Control button 120 may be configured to selectively allow and inhibit the pressurized fluid received via inlet 110 to flow through towards first channel 130 depending on an actuation state of control button 120, which is based on the presence of finger 20 thereon. For example, when finger 20 is positioned over and closing control button 120, control button 120 may be in an actuated state that is configured to direct the flow 60 of pressurized fluid received via inlet 110 into first opening 122 and through second opening 124 for delivery towards first channel 130. In other words, control button 120 may be configured to generate a vacuum and/or a sealed fluid connection within valve assembly 100, and particularly between inlet 110 and first channel 130, to thereby permit the flow 60 of pressurized fluid received in valve assembly 100 at inlet 110 from traveling through openings 122, 124 and into first channel 130 when finger 20 is disposed on control button 120.

By way of further example, when finger 20 is not positioned over and closing control button 120, control button 120 may be in an unactuated state that is configured to inhibit flow 60 of the pressurized fluid received via inlet 110 from traveling through inlet 110 and into first channel 130 via openings 122, 124 of control button 120. In other words, control button 120 may be configured to remove the vacuum and/or the sealed fluid connection within valve assembly 100 between inlet 110 and first channel 130. In this instance, control button 120 prevents flow 60 of the pressurized fluid from traveling through inlet 110 and through openings 122, 124 for receipt into first channel 130 when finger 20 is removed from control button 120. Stated differently, control button 120 may be open to an atmospheric pressure when finger 20 is not sealed thereon, thereby inhibiting creation of a vacuum between control button 120 and inlet 110 for propelling the pressurized fluid received from the fluid source from being urged through valve assembly 100.

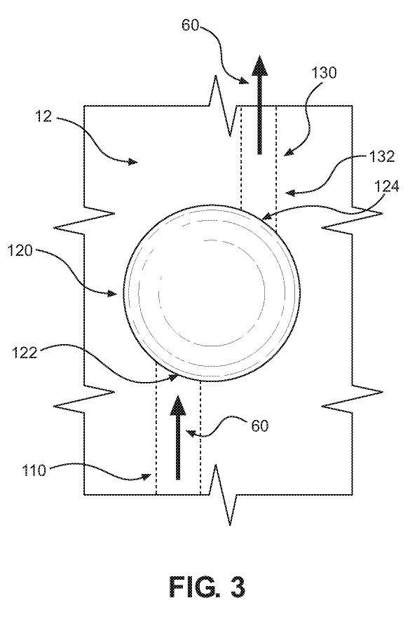

Referring now to FIG. 3, and as described above, control button 120 may be in fluid communication with inlet 110 via first opening 122 and first end 132 of first channel 130 via second opening 124. In some embodiments, first opening 122 and second opening 124 may be offset (e.g., laterally, longitudinally, etc.) relative to one another such that the pressurized fluid received from inlet 110 at first opening 122 may not be directly received into second opening 124. Stated differently, with openings 122, 124 misaligned with one another, the pressurized fluid received in control button 120 from first opening 122 may be encounter one or more interior surfaces defining control button 120 prior to receipt by second opening 124. In this instance, control button 120 may be sized, shaped, and/or otherwise configured to control a flow rate of the flow 60 of pressurized fluid entering first channel 130 from second opening 124 relative to an initial flow rate received by first opening 122 from inlet 110.

For example, control button 120 may be configured to limit and/or inhibit a momentum of the pressurized fluid received from inlet 110 via first opening 122 prior to the pressurized fluid exiting control button 120 and entering first channel 130 via second opening 124. In other words, with first opening 122 and second opening 124 arranged and/or oriented in an offset alignment with one another, control button 120 may be configured to direct the pressurized fluid received from inlet 110 against one or more interior surfaces of handle body 12 defining control button 120 prior to directing the pressurized fluid into first channel 130 and/or out via the opening defined on of handle body 12 at control button 120. Accordingly, control button 120 may be configured to inhibit delivery of the flow 60 of pressurized fluid received at first opening 122 from inlet 110 into first channel 130 via second opening 124 when finger 20 is removed from control button 120. In this instance, control button 120 may be configured to allow the pressurized fluid to exit out of control button 120 via the exposed opening on handle body 12 at control button 120.

As described herein, actuation of control button 120 via finger 20 (not shown) may seal control button 120 and permit the pressurized fluid received via inlet 110 to move towards second opening 124 and into first channel 130. Actuation of control button 120 via finger 20 (not shown) may partially and/or fully seal the opening on handle body 12 defined by control button 120 to maintain the pressurized fluid within valve assembly 100 for delivery towards enclosure 170 and/or outlet 190. As previously described, actuation of control button 120 may be performed based on the presence or absence of finger 20 (not shown).

In some embodiments, selective actuation of control button 120, such as controlled movements of finger 20, may be configured to control flow 60 of pressurized fluid through valve assembly 100. For example, control button 120 may be selectively actuated via one or more manual and/or physical taps, pulses, long presses, or other suitable contact that at least partially covers and/or seals control button 120 via finger 20 of the user to produce various delivery methods of the pressurized fluid through valve assembly 100 (e.g., one or more short fluid bursts, one or more long fluid flows, a single continuous fluid stream, and more.). Control button 120 may be configured to allow for fine-tuned adjustments of flow 60 of the pressurized fluid through valve assembly 100, such as a velocity, a volume, and various other parameters or characteristics of the pressurized fluid.

Referring back to FIG. 2, first channel 130 may be configured to receive the pressurized fluid from inlet 110 through control button 120. First channel 130 may be disposed within handle body 12 and include a first end 132, a second end 136 positioned opposite first end 132, and a junction 134 positioned between first end 132 and second end 136. First end 132 and second end 136 may define a longitudinal length of first channel 130. First end 132 may be positioned adjacent to and in fluid communication with second opening 124, and downstream from first opening 122. In this instance, first end 132 may be in fluid communication with inlet 110 via control button 120. Second end 136 may be positioned adjacent to and in fluid communication with a first opening 172 of enclosure 170, and downstream from second opening 124. In this instance, enclosure 170 may be in fluid communication with inlet 110 via first channel 130 through second end 136 and first opening 172. It should be appreciated that first channel 130, including each of first end 132, junction 134, and second end 136, may have various other suitable shapes, sizes, arrangements, and/or configurations than those shown and described herein without departing from a scope of this disclosure.

Second channel 140 may be configured to receive the pressurized fluid received in valve assembly 100 at inlet 110 through control button 120 and first channel 130. Second channel 140 may be disposed within handle body 12 and include a first end 142 and a second end 144 defining a longitudinal length of second channel 140. Second channel 140 may be sized, shaped, and/or otherwise configured to have an arrangement and/or configuration that extends around enclosure 170, thereby bypassing enclosure 170, and particularly first opening 172. First end 142 may be positioned adjacent to and in fluid communication with junction 134 of first channel 130, downstream from second opening 124 of control button 120. In this instance, first end 142 may be in fluid communication with inlet 110 via control button 120 and first channel 130. As described herein, second channel 140 may be configured to divert a portion of pressurized fluid received at first end 142 from junction 134 away from enclosure 170, such that the portion of pressurized fluid is not introduced into cavity 171 for mixing with agent 50 stored in enclosure 170.

Still referring to FIG. 2, second end 144 may be positioned adjacent to and in fluid communication with a third channel 155 of valve assembly 100 that is coupled to second channel 140 at second end 144. Third channel 155 may be fluid coupled to outlet 190. In this instance, second end 144 may be in fluid communication with outlet 190 via third channel 155. It should be appreciated that second channel 140, including each of first end 142 and second end 144, may have various other suitable shapes, sizes, arrangements, and/or configurations than those shown and described herein without departing from a scope of this disclosure. Additionally and/or alternatively, in other embodiments, third channel 155 may be omitted entirely such that second end 144 may be directly coupled to outlet 190 such that second channel 140 may be in fluid communication with outlet 190 without an intermediate channel disposed therebetween.

In the example, third channel 155 may include first valve 150. Stated differently, first valve 150 may be coupled to and/or positioned along third channel 155. In this instance, first valve 150 may be disposed downstream from one or more of first channel 130 and/or second channel 140 and upstream from outlet 190. First valve 150 may be configured to selectively allow and/or inhibit the pressurized fluid received at inlet 110 from the fluid source from traveling towards outlet 190 based on a configuration of first valve 150. In other embodiments in which third channel 155 is omitted entirely from valve assembly 100, first valve 150 may be positioned downstream from second channel 140 and upstream from outlet 190, such as adjacent to second end 144.

Still referring to FIG. 2, second valve 160 may be disposed downstream from one or more of enclosure 170 and/or first channel 130, and upstream from one or more of outlet 190 and/or third channel 155. Second valve 160 may be disposed within a fourth channel 165 of valve assembly 100 that is coupled to enclosure 170 at second opening 176. Fourth channel 165 may be fluid coupled to outlet 190. In this instance, second opening 176 may be in fluid communication with outlet 190 via fourth channel 165. Additionally and/or alternatively, in other embodiments, fourth channel 165 may be omitted entirely such that second opening 176 may be directly coupled to outlet 190 such that enclosure 170 may be in fluid communication with outlet 190 without an intermediate channel disposed therebetween.

In the example, fourth channel 165 may include second valve 160. Stated differently, second valve 160 may be coupled to and/or positioned along fourth channel 165. In this instance, second valve 160 may be disposed downstream from one or more of first channel 130 and/or enclosure 170 and upstream from outlet 190. Second valve 160 may be configured to selectively allow and/or inhibit a mixture combination of the pressurized fluid received in enclosure 170 from first channel 130 and agent 50 prestored in cavity 171 from traveling towards outlet 190 based on a configuration of second valve 160. In other embodiments in which fourth channel 165 is omitted entirely from valve assembly 100, second valve 160 may be positioned downstream from enclosure 170 and upstream from outlet 190, such as adjacent to second opening 176.

First valve 150 and second valve 160 may be actuated between a plurality of positions for selectively allowing and/or inhibiting the pressurized fluid and/or agent 50 from moving through valve assembly 100 via one or more of first channel 130, second channel 140, and/or enclosure 170. For example, one or more of first valve 150 and/or second valve 160 may be manually actuated to move between the plurality of positions. In other examples, one or more of first valve 150 and/or second valve 160 may be automatically actuated, such as in response to an actuation of delivery system 10, such as via actuation mechanism 30 (see FIG. 1). Although not shown, it should be appreciated that valves 150, 160 may be movably coupled to actuation mechanism 30, such as via one or more mechanical linkages.

Still referring to FIG. 2, first valve 150 and second valve 160 may be configured to move independently from one another between the plurality of positions, respectively, for selectively delivering and/or inhibiting one or more of the pressurized fluid and/or agent 50. In some embodiments, first valve 150 and/or second valve 160 may include a plurality of predetermined discrete set points / positions configured to allow different amounts and/or flow rates of the pressurized fluid and/or mixture of fluid and agent 50 to be delivered out of valve assembly 100. Although not shown, it should be appreciated that valve assembly 100 may include corresponding indicia on valves 150, 160 for each of the plurality of predetermined discrete set points / positions.

Outlet 190 may be fluidly coupled to third channel 155 such that outlet 190 may be configured to receive pressurized fluid in isolation from agent 50 from third channel 155 via channels 130, 140. Outlet 190 may be fluidly coupled to fourth channel 165 such that outlet 190 may be configured to receive a combination of pressurized fluid and agent 50 from fourth channel 165 via enclosure 170. In some embodiments, valve assembly 100 may include a connector 192 positioned downstream from valves 150, 160 and upstream from outlet 190. Connector 192 may be configured to selectively allow and/or inhibit delivery of the pressurized fluid from third channel 155 and/or the combination of fluid and agent 50 from fourth channel 165 depending on a position (e.g., open, closed, etc.) of connector 192. It should be appreciated that connector 192 may be actuated via one or more suitable mechanisms or means as valves 150, 160 described above.

In exemplary use, valve assembly 100 may be configured to selectively allow and/or inhibit the flow 60 of pressurized fluid in response to the actuation of control button 120 via finger 20. For example, valve assembly 100 may be configured to inhibit the flow 60 of the pressurized fluid through valve assembly 100, and particularly inlet 110, when finger 20 is not at least partially disposed over control button 120. In other words, when finger 20 is not positioned on, covering / sealing (e.g., partially or completely) over, or otherwise closing control button 120, the flow 60 of the pressurized fluid may be inhibited from traveling through inlet 110 and into first channel 130 via control button 120. In some embodiments, with control button 120 uncovered / unsealed by finger 20, any pressurized fluid received through inlet 110 may exit handle body 12 via control button 120, rather than flowing through towards first channel 130.

When finger 20 is at least partially positioned on control button 120, such that finger 20 covers at least a portion of control button 120, control button 120 may be configured to guide the flow 60 of pressurized fluid from inlet 110 through openings 122, 124 and into first channel 130. It should be appreciated that an amount of pressurized fluid and/or a flow rate of the flow 60 of pressurized fluid received in first channel 130 from inlet 110 may be selectively adjustable based on an extent of closure of control button 120 by finger 20. For example, control button 120 may be configured and operable such that the amount of pressurized fluid and/or flow rate of the flow 60 of pressurized fluid delivered through inlet 110 and towards first channel 130 may be relatively greater when control button 120 is completely sealed by finger 20 relative to when control button 120 is partially sealed by finger 20.

Still referring to FIG. 2, first channel 130 may be configured such that the flow 60 of pressurized fluid received therein may be at least partially separated, divided, and/or distributed into multiple flow paths via junction 134, such as towards enclosure 170 and second channel 140. In this instance, junction 134 may be configured to guide a first portion of the pressurized fluid received in first channel 130 towards second end 136 and into cavity 171 of enclosure 170 via first opening 172.

First opening 172 may be configured to direct the first portion of pressurized fluid into cavity 171 for moving, mixing, and/or agitating agent 50 within enclosure 170. In other words, first channel 130 may be configured to facilitate the first portion of the pressurized fluid creating a swirl or vortex within cavity 171 for agitating agent 50 in enclosure 170, thereby assisting in mixing the first portion of pressurized fluid with agent 50. In some examples, second end 136 of first channel 130 may be sized, shaped, and/or otherwise configured in an orientation and/or position relative to first opening 172 to urge the first portion of pressurized fluid into enclosure 170 at an angle and/or arrangement that facilitates creation of the swirl or vortex in cavity 171.

Junction 134 may be configured to guide a second portion of the pressurized fluid received in first channel 130 into second (bypass) channel 140 via first end 142 of second channel 140 that is in fluid communication with first channel 130 at junction 134. Second channel 140 may be configured to direct the second portion of pressurized fluid received at first end 142 from junction 134 towards outlet 190 via second end 144 of second channel 140, thereby bypassing enclosure 170. Second end 144 may be fluidly coupled to third channel 155, such that second channel 140 may be configured to deliver the second portion of pressurized fluid into third channel 155. In this instance, second (bypass) channel 140 may be configured to deliver the second portion of pressurized fluid to bypass mixing with agent 50 stored in enclosure 170.

First valve 150 may be configured to selectively control a release of the second portion of pressurized fluid through third channel 155 and into outlet 190 based on a state and/or position of first valve 150. When in a closed state and/or position, first valve 150 may be configured to inhibit delivery of the second portion of pressurized fluid out of third channel 155 and through outlet 190. When in an open state and/or position, first valve 150 may be configured to deliver the second portion of pressurized fluid out of third channel 155 and through outlet 190, such as in isolation from and/or in combination with agent 50 from enclosure 170 based on a state and/or position of second valve 160.

Second valve 160 may be configured to selectively control a release of the mixture of pressurized fluid and agent 50 through fourth channel 165 and into outlet 190 based on a state and/or position of first valve 150. When in a closed state and/or position, second valve 160 may be configured to inhibit delivery of the mixture out of fourth channel 165 and through outlet 190. When in an open state and/or position, second valve 160 may be configured to deliver the mixture out of fourth channel 165 and through outlet 190, such as in isolation from and/or in combination with the second portion of pressurized fluid from third channel 155 based on a state and/or position of first valve 150.

Still referring to FIG. 2, valve assembly 100 may be configured to combine, group, and/or otherwise mix the combination of pressurized fluid and agent 50 from enclosure 170 with the pressurized fluid from second channel 140 through outlet 190 when each of first valve 150 and second valve 160 are in the open state and/or position. First valve 150 may be configured to deliver the second portion of pressurized fluid through outlet 190 for preventing clogging or build up of residual agent 50 within outlet 190 or a delivery conduit coupled thereto (e.g., catheter 36), such as before, during, and/or after delivery of the mixture of pressurized fluid and agent 50 from enclosure 170.

In other words, first valve 150 may be configured to provide insufflation through outlet 190 with the second portion of the pressurized fluid received from second channel 140, such as prior to receipt of the mixture from enclosure 170 into outlet 190, during simultaneous receipt of the mixture from enclosure 170 into outlet 190, and/or completion of receipt of the mixture from enclosure 170 into outlet 190. First valve 150 may be further configured to deliver the second portion of pressurized fluid through outlet 190 during simultaneous delivery of the mixture from enclosure 170 to facilitate and/or assist in delivering the mixture with sufficient flow rate through outlet 190.

In some embodiments, valve assembly 100 may be configured to generate feedback (e.g., tactile) at control button 120 that is indicative of an occurrence of clogging downstream, such as at fourth channel 165, outlet 190, and/or the delivery conduit coupled to outlet 190. For example, control button 120 may be configured to provide tactile feedback against finger 20 of the user’s hand with the pressurized fluid passing therethrough when a buildup of agent 50 accumulates downstream from control button 120.

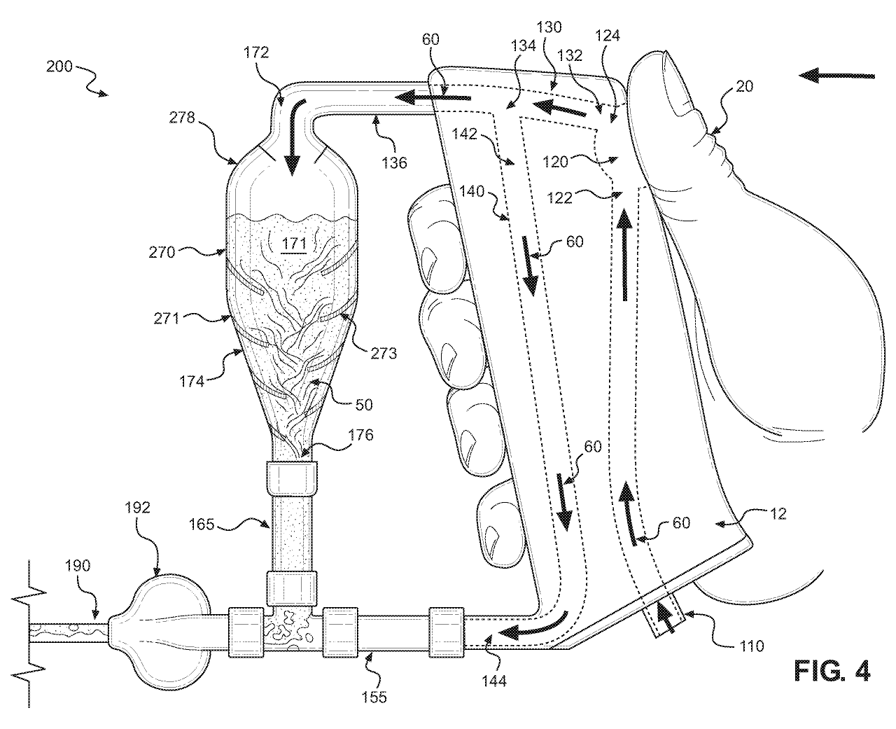

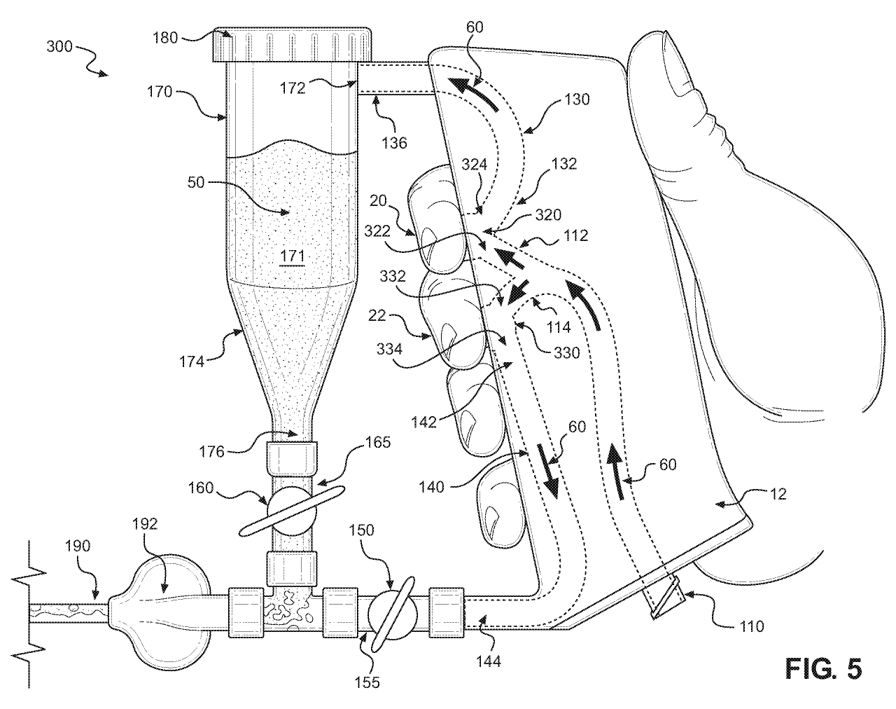

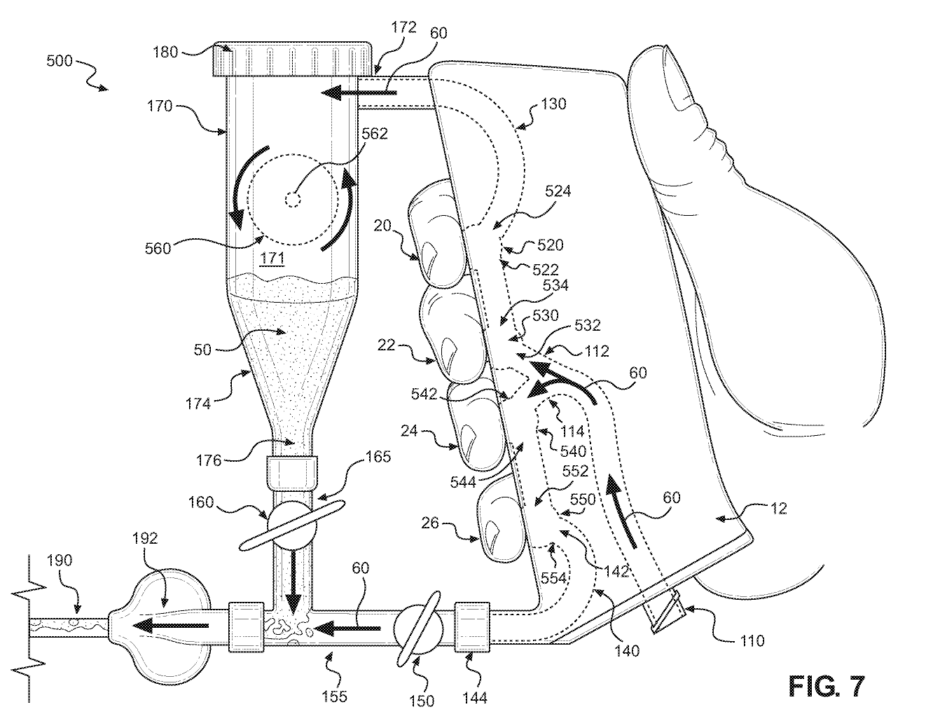

It should be appreciated that although valve assembly 100 is shown and described above as including a single control button 120 and a pair of valves 150, 160, valve assembly 100 may be configured and operable to include additional and/or fewer control buttons (see FIGS. 5 and 7) and/or valves (see FIG. 4) without departing from a scope of this disclosure. Additionally, valve assembly 100, control button 120, and valves 150, 160 may be configured and operable to move towards any one of the discrete positions and/or configurations described above in various other sequences of operation than the arrangement described above without departing from a scope of this disclosure.

FIG. 4 shows another exemplary valve assembly 200, according to one or more embodiments. Valve assembly 200 may be part of delivery system 10 as discussed with reference to FIG. 1 above, such as fluidly coupled to enclosure 14. It should be appreciated that valve assembly 200 may be configured and operable like valve assembly 100 shown and described above except for the differences explicitly noted herein. Accordingly, similar reference numerals are used to identify similar components.

Valve assembly 200 may include a chamber and/or enclosure 270 for storing a material, such as agent 50 (e.g. a powdered agent). A predetermined amount of agent 50 may be stored within enclosure 270 for delivery from valve assembly 200. The predetermined amount of agent 50 stored therein may vary depending on a desired application of use, such as a treatment of a subject (e.g., patient) or a target treatment site. Enclosure 270 may be in fluid communication with inlet 110 and outlet 190 via first channel 130 and fourth channel 165, respectively. Enclosure 270 may include an interior surface 271 and a second funnel 278 collectively defining cavity 171 for storing a material, e.g., agent 50.

Still referring to FIG. 4, first opening 172 may be positioned upstream from second funnel 278. Second funnel 278 may be sized, shaped, and/or otherwise configured to direct the first portion of pressurized fluid received in enclosure 270 via first opening 172 into cavity 171 and towards agent 50 stored therein. Second funnel 278 may be positioned relative to first opening 172 such as to facilitate the first portion of the pressurized fluid in creating a swirl or vortex within cavity 171 for agitating agent 50 stored within enclosure 270, thereby assisting in mixing the first portion of pressurized fluid and agent 50. One or more of (first) funnel 174 and second funnel 278 may be configured in a tapered, a conical, and/or an angled shape to assist agent 50 in moving within enclosure 270.

Enclosure 270 may include a plurality of agitation mechanisms 273 disposed within cavity 171. In the example, the plurality of agitation mechanisms 273 may be disposed along and fixed to interior surface 271, such as a sidewall of enclosure 270. For example, agitation mechanisms 273 may be sized, shaped, and/or otherwise configured to extend radially inwards from interior surface 271 and into cavity 171. It should be appreciated that agitation mechanisms 273 may include a physical impediment or structural obstruction for a free movement of pressurized fluid, agent 50, and/or a combination thereof in cavity 171. For example, agitation mechanisms 273 may include one or more ribs, protrusions, tabs, steps, flanges, ramps, and/or other suitable surfaces suspended within cavity 171 for contacting and/or abutting against the collective material inside enclosure 170. The plurality of agitation mechanisms 273 may be configured to facilitate agitating, mixing, moving, and/or combining the pressurized fluid and agent 50 within enclosure 270.

It should be appreciated that additional and/or fewer agitation mechanisms 273 may be included in enclosure 270 without departing from a scope of this disclosure. Additionally and/or alternatively, one or more of the plurality of agitation mechanisms 273 may be disposed along various other suitable surfaces of enclosure 270, such as along funnels 174, 278. Although agitation mechanisms 273 are shown and described herein with respect to valve assembly 200, it should be appreciated that agitation mechanisms 273 may be incorporated into any of the other valve assemblies described herein without departing from a scope of this disclosure. In the example, valve assembly 200 omits valves 150, 160. However, it should be understood that valve assembly 200 may include one or more valves 150, 160 without departing from a scope of this disclosure.

In exemplary use, enclosure 270 may be configured such that the pressurized fluid received at first opening 172 may be directed downwards towards agent 50 via second funnel 278. Agitation mechanisms 273 may be disposed within cavity 171 between first opening 172 and second opening 176 such that the pressurized fluid directed into cavity 171 encounters agitation mechanisms 273 prior to exiting enclosure 270. Agitation mechanisms 273 may be configured to facilitate a mixture of the pressurized fluid with agent 50 prior to a release of the mixture through second opening 176 and into outlet 190.

FIG. 5 shows another exemplary valve assembly 300, according to one or more embodiments. Valve assembly 300 may be part of delivery system 10 as discussed with reference to FIG. 1 above, such as fluidly coupled to enclosure 14. It should be appreciated that valve assembly 300 may be configured and operable like valve assemblies 100, 200 shown and described above except for the differences explicitly noted herein. Accordingly, similar reference numerals are used to identify similar components.

Inlet 110 of valve assembly 300 may include a first branch 112 and a second branch 114. Valve assembly 300 may further include a first control button 320 (e.g., a first actuator) and a second control button 330 (e.g., a second actuator) disposed along handle body 12, each of which may be in fluid communication with at least one of branches 112, 114 of inlet 110. First control button 320 and second control button 330 may be configured and operable similar to control button 120 shown and described in detail above. For example, each of control buttons 320, 330 may include an actuator that is sized, shaped, and/or otherwise configured as an opening positioned along handle body 12 for selective actuation by a user, and particularly a hand of the user.

Stated differently, each of control buttons 320, 330 may include an opening, a recess, a cavity, an aperture, and/or an indentation on handle body 12 that is each configured to at least partially receive a portion of the hand of a user, such as within control buttons 320, 330, over control buttons 320, 330, or a combination thereof. In other words, control buttons 320, 330 may be manually accessible by the user from an exterior of handle body 12. As described in detail herein, control buttons 320, 330 may be in fluid communication with one or more channels, inlets, and/or outlets of valve assembly 300.

Still referring to FIG. 5, control buttons 320, 330 may each be configured to control the flow 60 of pressurized fluid through valve assembly 300 via selective actuation by at least one finger of the user’s hand. In the example, first control button 320 may be positioned relatively along handle body 12 to receive at least one finger 20 of the user’s hand and second control button 330 may be positioned relatively along handle body 12 to receive at least another finger 22 of the user’s hand during use.

Control buttons 320, 330 may be configured to transition to a plurality of configurations upon actuation by a user, such as a hand of the user. In the example, first control button 320 may be actuated independently and/or simultaneously with second control button 330, and vice versa. As described herein, each of control buttons 320, 330 may be configured to selectively guide a portion of pressurized fluid received in valve assembly 300 via inlet 110 to one or more components of valve assembly 300, such as enclosure 170 and outlet 190, respectively.

Still referring to FIG. 5, first control button 320 may include a first opening 322 and a second opening 324 positioned along an opposing surface of first control button 320 relative to first opening 322. First opening 322 may be positioned downstream from first branch 112 of inlet 110 and second opening 324 may be positioned upstream from first channel 130. First opening 322 may be configured to receive the first portion of pressurized fluid received via first branch 112 of inlet 110, and second opening 324 may be configured to guide the first portion of pressurized fluid received in first control button 320 towards enclosure 170 via first channel 130 when finger 20 is positioned over first control button 320.

Second control button 330 may include a first opening 332 and a second opening 334 positioned along an opposing surface of second control button 330 relative to first opening 332. First opening 332 may be positioned downstream from second branch 114 of inlet 110, and second opening 334 may be positioned upstream from second channel 140. First opening 332 may be configured to receive the second portion of pressurized fluid received via second branch 114 of inlet 110, and second opening 334 may be configured to guide the second portion of pressurized fluid received in second control button 330 towards outlet 190 via second channel 140 when finger 22 is positioned over second control button 330.

In the example, first control button 320 may be configured to selectively deliver the pressurized fluid from inlet 110 towards enclosure 170 for agitating agent 50 stored therein independently from a delivery of pressurized fluid from inlet 110 towards outlet 190 in isolation from agent 50. Further, second control button 330 may be configured to selectively deliver the pressurized fluid from inlet 110 towards outlet 190 for flushing the delivery conduit (e.g. catheter 36) coupled thereto independently from a delivery of agent 50 from enclosure 170 towards outlet 190.

In exemplary use, first control button 320 may be configured to deliver the first portion of pressurized fluid received from first branch 112 of inlet 110 towards first channel 130 when finger 20 is disposed atop first control button 320. The flow 60 of pressurized fluid may be guided through first channel 130 and towards first opening 172 for entry into enclosure 170 and mixture with agent 50 stored in cavity 171. First control button 320 may be further configured to inhibit delivery of the first portion of pressurized fluid received from first branch 112 towards first channel 130 when finger 20 is removed from contacting first control button 320. For example, when finger 20 is not secured against first control button 320, valve assembly 300 may be configured to release the pressurized fluid received from first branch 112 of inlet 110 to a surrounding atmosphere of handle body 12 via the opening of first control button 320.

Second control button 330 may be configured to deliver the second portion of pressurized fluid received from second branch 114 of inlet 110 towards second channel 140 when finger 22 is disposed atop second control button 330. The flow 60 of pressurized fluid may be guided through second channel 140 and towards second end 144 for delivery out of outlet 190 via first valve 150. Second control button 330 may be further configured to inhibit delivery of the second portion of pressurized fluid received from second branch 114 towards second channel 140 when finger 22 is removed from contacting second control button 330. For example, when finger 22 is not secured against second control button 330, valve assembly 300 may be configured to release the pressurized fluid received from second branch 114 of inlet 110 to a surrounding atmosphere of handle body 12 via the opening of second control button 330.

It should be understood that when both fingers 20, 22 are positioned relative to handle body 12 to conceal the openings of control buttons 320, 330, respectively, valve assembly 300 may be configured to guide the first and second portions of pressurized fluid from inlet 110 simultaneously towards enclosure 170 and outlet 190, respectively.

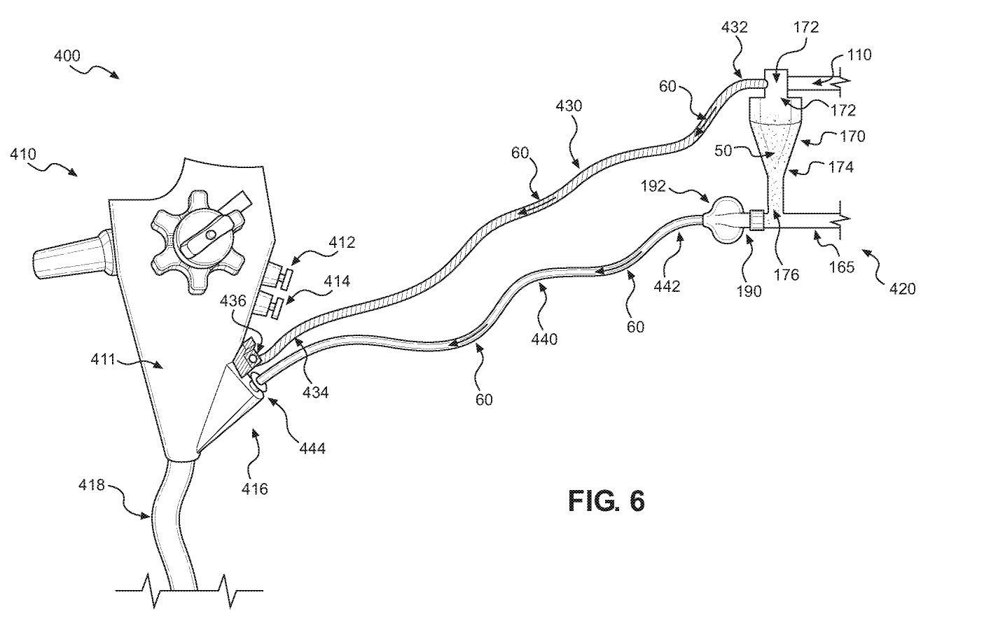

FIG. 6 shows an exemplary medical system 400 including a medical device 410 and a valve assembly 420, according to one or more embodiments. Medical device 410 may include a scope, including but not limited to, a gastroscope, an endoscope, etc. In the example, medical device 410 may include a handle 411 with a first actuator 412, a second actuator 414, and a port 416 disposed along handle 411. Medical device 410 may further include a delivery conduit 418 (e.g., a catheter, a shaft, a tube, etc.) fluidly coupled to handle 411. Port 416 may include one or more openings that are in fluid communication with one or more corresponding channels (not shown) disposed in handle 411 that extend into delivery conduit 418. Although not shown, it should be appreciated that the one or more channels may terminate at a distal end of delivery conduit 418. As described in detail herein, medical device 410 may be configured to fluidly connect to valve assembly 420, and valve assembly 420 may be configured to deliver pressurized fluid and/or agent 50 to medical device 410 for delivery to a target treatment site via delivery conduit 418.

Valve assembly 420 may be part of delivery system 10 as discussed with reference to FIG. 1 above, such as fluidly coupled to enclosure 14. In this instance, medical device 410 may be a first device of medical system 400 and delivery system 10 including valve assembly 420 may be a second device of medical system 400. It should be appreciated that valve assembly 420 may be configured and operable like valve assemblies 100, 200, 300 shown and described above except for the differences explicitly noted herein. Accordingly, similar reference numerals are used to identify similar components.

Valve assembly 420 may include inlet 110 that is fluidly coupled to enclosure 170 via first opening 172 and a first conduit 430. First conduit 430 may be further fluidly coupled to medical device 410 via at least one of the openings on port 416, such that first conduit 430 may be configured to deliver the pressurized fluid received from inlet 110 towards medical device 410 (in isolation from agent 50). First conduit 430 may include a first end 432 and a second end 434 defining a longitudinal length of first conduit 430. First end 432 may be coupled to and in fluid communication with inlet 110, and second end 434 may be attached to handle 411, such as adjacent to port 416. In the example, second end 434 may include a fastening mechanism that is configured to secure first conduit 430 to an exterior portion of handle 411, thereby facilitating access to second end 434 by a hand of a user of medical system 400. As described herein, second end 434 may include an actuator (e.g., control button) for selective actuation by the user during handling of medical device 410.

Still referring to FIG. 6, valve assembly 420 may include a control button 436 coupled to first conduit 430. In the example, control button 436 may be positioned along second end 434 such that control button 436 may be disposed adjacent to port 416 when second end 434 is fluidly coupled to medical device 410 at port 416. Control button 436 may be substantially similar to control buttons 120, 320, 330 as shown and described above. Accordingly, control button 436 may include an opening at second end 434 that may be selectively actuated by a hand of a user, such as a finger. As described in detail herein, control button 436 may be configured to selectively control a delivery of agent 50 and pressurized fluid from valve assembly 420 towards medical device 410 in response to actuation of control button 436.

Valve assembly 420 may further include a second conduit 440 that is fluidly coupled to each of outlet 190 and medical device 410 via at least one of the openings on port 416, such that second conduit 440 may be configured to deliver the mixture of agent 50 and pressurized fluid received from enclosure 170 through outlet 190 and towards medical device 410. Second conduit 440 may include a first end 442 and a second end 444 defining a longitudinal length of second conduit 440. First end 442 may be coupled to and in fluid communication with outlet 190 via connector 192, and second end 444 may be coupled to and in fluid communication with at least one of the openings of port 416.

Still referring to FIG. 6, although not shown it should be appreciated that valve assembly 420 may include one or more of first channel 130, second channel 140, first valve 150, and/or second valve 160 as shown and described above. For example, valve assembly 420 may include second channel 140 that fluidly couples third channel 155 with inlet 110 for delivering a portion of pressurized fluid towards outlet 190 to facilitate delivery of the mixture of agent 50 and pressurized fluid released from enclosure 170 at second opening 176 through second conduit 440.

First actuator 412 of medical device 410 may be configured to activate flow of a pressurized fluid (e.g., gas, air, liquid, etc.) from a pressurized fluid source (not shown) that is in fluid communication with medical device 410. In some embodiments, the pressurized fluid source may be the same as that fluidly coupled to inlet 110 of valve assembly 420, while in other embodiments the pressurized fluid source of medical device 410 may be separate from that of valve assembly 420. First actuator 412 may be selectively actuated (e.g., manually depressible) or otherwise moved to control delivery of the pressurized fluid through the one or more channels in handle 411 and delivery conduit 418. Second actuator 414 may be configured to activate suction and/or generate a vacuum through the one or more channels in handle 411 and delivery conduit 418.

In exemplary use, valve assembly 420 may be actuated to initiate delivery of pressurized fluid through inlet 110. With control button 426 in an unactuated state, such as when a finger of the user’s hand is not positioned thereon, first conduit 430 may be configured to receive the flow 60 of pressurized fluid at first end 432 for release via the opening of control button 436 at second end 434. It should be appreciated that control button 436 may be configured to divert the pressurized fluid from inlet 110 into first end 432 of first conduit 430 and away from first opening 172 of enclosure 170 when control button 436 is not actuated.

When control button 436 is an actuated state, valve assembly 420 may be configured to guide the pressurized fluid from inlet 110 through first opening 172 and into enclosure 170 to mix, move, combine, and/or agitate agent 50 stored therein. In other words, control button 436 may be configured to divert the pressurized fluid away from entering first conduit 430 at first end 432 and entering enclosure 170 at first opening 172. Funnel 174 may be configured to direct the mixture of agent 50 and pressurized fluid towards second opening 176 for receipt at third channel 155. With second conduit 440 fluidly coupled to outlet 190 via connector 192, valve assembly 420 may be configured to urge the mixture into second conduit 440 at first end 442 for delivery into medical device 410 at second end 444 that is fluidly coupled to port 416. Stated differently, second conduit 440 may be configured to receive the mixture of the pressurized fluid and agent 50 exiting enclosure 170 via outlet 190, and direct the mixture to medical device 410 for delivery into delivery conduit 418.

It should be appreciated that although valve assembly 420 is shown and described above as including a single control button 436, valve assembly 420 may be configured and operable to include additional control buttons without departing from a scope of this disclosure. Alternatively, although control button 436 is shown and described herein as being positioned adjacent to second end 434 of first conduit 430, it should be appreciated that control button 436 may be positioned along various other ends and/or portions of first conduit 430, and/or other components of valve assembly 420, without departing from a scope of this disclosure.

FIG. 7 shows another exemplary valve assembly 500, according to one or more embodiments. Valve assembly 500 may be part of delivery system 10 as discussed with reference to FIG. 1 above, such as fluidly coupled to enclosure 14. It should be appreciated that valve assembly 500 may be configured and operable like valve assemblies 100, 200, 300 shown and described above except for the differences explicitly noted herein. Accordingly, similar reference numerals are used to identify similar components.

Inlet 110 of valve assembly 500 may include first branch 112 and second branch 114. Valve assembly 500 may include a plurality of control buttons disposed along handle body 12, including at least a first control button 520, a second control button 530, a third control button 540, and a fourth control button 550. Each of control buttons 520, 530, 540, 550 may be in direct and/or indirect fluid communication with at least one of branches 112, 114 of inlet 110. As described herein, first control button 520 and second control button 530 may collectively define a first set / pair of control buttons that are collectively configured to deliver a first portion of pressurized fluid from inlet 110 towards enclosure 170. Further, third control button 540 and fourth control button 550 may collectively define a second set / pair of control buttons that are collectively configured to deliver a second portion of pressurized fluid from inlet 110 towards outlet 190 while bypassing enclosure 170.

Control buttons 520, 530, 540, 550 may be configured and operable similar to control button 120 shown and described in detail above. For example, each of control buttons 520, 530, 540, 550 may include an actuator that is sized, shaped, and/or otherwise configured as an opening positioned along handle body 12 for selective actuation by a user, and particularly a hand of the user.

Stated differently, each of control buttons 520, 530, 540, 550 may include an opening, a recess, a cavity, an aperture, and/or an indentation on handle body 12 that is each configured to at least partially receive a portion of the hand of a user, such as within control buttons 520, 530, 540, 550, over control buttons 520, 530, 540, 550, or a combination thereof. In other words, control buttons 520, 530, 540, 550 may be manually accessible by the user from an exterior of handle body 12. As described in detail herein, control buttons 520, 530, 540, 550 may be in fluid communication with one or more channels, inlets, outlets of valve assembly 300, and/or one another.

Still referring to FIG. 7, control buttons 520, 530, 540, 550 may each be configured to control the flow 60 of pressurized fluid through valve assembly 500 via selective actuation by at least one finger of the user’s hand. In the example, first control button 520 may be positioned relatively along handle body 12 to receive at least a first finger 20 of the user’s hand, second control button 530 may be positioned relatively along handle body 12 to receive at least a second finger 22 of the user’s hand, third control button 540 may be positioned relatively along handle body 12 to receive at least a third finger 24 of the user’s hand, and fourth control button 550 may be positioned relatively along handle body 12 to receive at least a fourth finger 24 of the user’s hand.

Control buttons 520, 530, 540, 550 may be configured to transition to a plurality of configurations upon actuation by a user, such as a hand of the user. In the example, first control button 520 may be actuated independently and/or simultaneously with second control button 530, and vice versa. Additionally, third control button 540 may be actuated independently and/or simultaneously with fourth control button 550, and vice versa. As described herein, each of control buttons 520, 530, 540, 550 may be configured to selectively guide a portion of pressurized fluid received in valve assembly 500 via inlet 110 to one or more components of valve assembly 500, such as enclosure 170 and outlet 190, respectively.

Still referring to FIG. 7, first control button 520 may include a first opening 522 and a second opening 524 positioned along opposing surfaces of first control button 520. First opening 522 may be positioned downstream from second control button 530, and second opening 524 may be positioned upstream from first channel 130. First opening 522 may be configured to receive the first portion of pressurized fluid received by second control button 530 via first branch 112. Second opening 524 may be configured to guide the first portion of pressurized fluid received in first control button 520 towards enclosure 170 when first finger 20 is sealed against first control button 520.

Second control button 530 may include a first opening 532 and a second opening 534 positioned along opposing surfaces of second control button 530. First opening 532 may be positioned downstream from first branch 112 and second opening 534 may be positioned upstream from first control button 520. First opening 532 may be configured to receive the first portion of pressurized fluid received via first branch 112 of inlet 110, and second opening 534 may be configured to guide the first portion of pressurized fluid received in second control button 530 towards first control button 520 via an intermediate channel disposed therebetween when second finger 22 is sealed against second control button 530.

It should be appreciated that valve assembly 500 may be configured such that both first control button 520 and second control button 530 are to be actuated to guide the first portion of pressurized fluid from first branch 112 into first channel 130 for delivery towards enclosure 170. In the example, valve assembly 500 may be operable to require simultaneous actuation of control buttons 520, 530 to agitate agent 50 within enclosure 170.

Still referring to FIG. 7, third control button 540 may include a first opening 542 and a second opening 544 positioned along opposing surfaces of third control button 540. First opening 542 may be positioned downstream from second branch 114 of inlet 110, and second opening 544 may be positioned upstream from fourth control button 550. First opening 542 may be configured to receive the second portion of pressurized fluid received via second branch 114 of inlet 110, and second opening 544 may be configured to guide the second portion of pressurized fluid received in third control button 540 towards fourth control button 550 via an intermediate channel disposed therebetween when third finger 24 is sealed against third control button 540.

Fourth control button 550 may include a first opening 552 and a second opening 554 positioned along opposing surfaces of fourth control button 550. First opening 552 may be positioned downstream from third control button 540 and second opening 554 may be positioned upstream from second channel 140. First opening 552 may be configured to receive the second portion of pressurized fluid from third control button 530, and second opening 554 may be configured to guide the second portion of pressurized fluid received in fourth control button 550 towards second channel 140 when fourth finger 26 is sealed against fourth control button 550.

It should be appreciated that valve assembly 500 may be configured such that both third control button 540 and fourth control button 550 are to be actuated to guide the second portion of pressurized fluid from second branch 114 into second channel 140 for delivery towards outlet 190. In the example, valve assembly 500 may be operable to require simultaneous actuation of control buttons 540, 550 to flush outlet 190 with pressurized fluid in isolation of agent 50.

In the example, control buttons 520, 530 may be collectively configured to selectively deliver the pressurized fluid from inlet 110 towards enclosure 170 for agitating agent 50 stored therein independently from a delivery of pressurized fluid from inlet 110 towards outlet 190 in isolation from agent 50. Further, control buttons 540, 550 may be collectively configured to selectively deliver the pressurized fluid from inlet 110 towards outlet 190 for flushing the delivery conduit (e.g. catheter 36) coupled thereto independently from a delivery of agent 50 from enclosure 170 towards outlet 190.

Still referring to FIG. 7, valve assembly 500 may include one or more agitation mechanisms 560 disposed within enclosure 170. In the example, agitation mechanism 560 may include a movable (rotatable) disc, wheel, rotator, and/or dial that may be configured to facilitate agitating, mixing, moving, and/or combining the pressurized fluid received in cavity 171 with agent 50 stored in enclosure 170. Agitation mechanism 560 may be rotatably coupled within enclosure 170, such as to an interior surface of enclosure 170 defining cavity 171. For example, agitation mechanism 560 may include a pin 562 coupled to the interior surface of enclosure 170, and agitation mechanism 560 may be sized, shaped, and/or otherwise configured to rotate about pin 562.

In some embodiments, agitation mechanism 560 may be configured to move in response to the pressurized fluid received in enclosure 170 at first opening 172. In this instance, the pressurized fluid entering cavity 171 may encounter agitation mechanism 560, thereby urging agitation mechanism 560 to rotate (e.g., in a clockwise direction, a counterclockwise direction, etc.) to facilitate agitating agent 50. In other embodiments, agitation mechanism 560 may be configured to move in response to an actuation of delivery system 10, such as agitation mechanism 30 (see FIG. 1). Although agitation mechanism 560 is shown and described herein with respect to valve assembly 500, it should be appreciated that agitation mechanism 560 may be incorporated into any of the other valve assemblies described herein without departing from a scope of this disclosure.

In exemplary use, control buttons 520, 530 may be collectively configured to deliver the first portion of pressurized fluid received from first branch 112 of inlet 110 towards first channel 130 when finger 20 is secured against handle body 12 at first control button 520 and finger 22 is secured against handle body 12 at second control button 530. It should be appreciated that each of first control button 520 and second control button 530 are actuated by fingers 20, 22 to fluidly couple inlet 110 with first channel 130. The flow 60 of pressurized fluid may be guided through first channel 130 and towards first opening 172 for entry into enclosure 170 and mixture with agent 50 stored in cavity 171.

Still referring to FIG. 7, agitation mechanism 560 may be configured to move (e.g., rotate) within cavity 171 during receipt of the pressurized fluid into enclosure 170 via first opening 172 to facilitate, enhance, and/or generate a mixture of the pressurized fluid and agent 50. Agitation mechanism 560 may be further configured to direct the mixture through cavity 171 and towards funnel 174 for delivery out of enclosure 170 via second opening 176. In some embodiments, agitation mechanism 560 may be configured to generate the swirl or vortex of agent 50 and pressurized fluid within cavity 171 prior to delivery of the mixture from enclosure 170.

Control buttons 520, 530 may be further configured to inhibit delivery of the first portion of pressurized fluid received from first branch 112 towards first channel 130 when at least one of fingers 20, 22 is removed from contacting the respective control button 520, 530. For example, when one of fingers 20, 22 is not secured against the respective control button 520, 530, valve assembly 500 may be configured to release the pressurized fluid received from first branch 112 of inlet 110 to a surrounding atmosphere of handle body 12 via the opening of the corresponding control button 520, 530 that is not actuated. In this instance, each of control buttons 520, 530 may provide a secondary actuator for valve assembly 500 that may serve as a safety feature to ensure the pressurized fluid is delivered only when both control buttons 520, 530 are actuated by the user.

Still referring to FIG. 7, control buttons 540, 550 may be collectively configured to deliver the second portion of pressurized fluid received from second branch 114 of inlet 110 towards second channel 140 when finger 24 is secured against handle body 12 at third control button 540 and finger 26 is secured against handle body 12 at fourth control button 550. It should be appreciated that each of at third control button 540 and fourth control button 550 are actuated by fingers 24, 26 to fluidly couple inlet 110 with second channel 140. The flow 60 of pressurized fluid may be guided through second channel 140 and towards third channel 155 for delivery out of valve assembly 500 via outlet 190.

Control buttons 540, 550 may be further configured to inhibit delivery of the second portion of pressurized fluid received from second branch 114 towards second channel 140 when at least one of fingers 24, 26 is removed from contacting the respective control button 540, 550. For example, when either finger 24, 26 is not secured against the respective control button 540, 550, valve assembly 500 may be configured to release the pressurized fluid received from second branch 114 of inlet 110 to a surrounding atmosphere of handle body 12 via the opening of the corresponding control button 540, 550 that is not actuated. In this instance, each of control buttons 540, 550 may provide a secondary actuator for valve assembly 500 that may serve as a safety feature to ensure the pressurized fluid is delivered only when both control buttons 540, 550 are actuated by the user.

Valve assembly 500 may be configured and operable to control the flow 60 of pressurized fluid via various suitable delivery methods in response to actuation of control buttons 520, 530, 540, 550 by fingers 20, 22, 24, 26. For example, fingers 20, 22, 24, 26 may be moved to partially cover, tap, long press, or a combination thereof to deliver the pressurized fluid and/or the mixture of agent 50 and pressurized fluid via one or more of short bursts, one or more long fluid flows, a single continuous fluid stream, and more. Control buttons 520, 530, 540, 550 may be configured to collectively provide for fine-tuned adjustments of the pressurized fluid and agent 50 delivery through valve assembly 500 in response to independently controlling and/or actuating each of control buttons 520, 530, 540, 550. For example, one or more of control buttons 520, 530, 540, 550 may be partially covered / sealed by the respective finger 20, 22, 24, 26 of the user while one or more of the other control buttons 520, 530, 540, 550 may be completely covered / sealed to selectively adjust an amount and/or a flow rate of the pressurized fluid delivered through valve assembly 500.

Although valve assemblies 100, 200, 300, 500 are shown and described herein as being implemented in delivery system 10, and particularly in a handheld device, it should be appreciated that they may be configured and operable in various other suitable configurations. For example, valve assemblies 100, 200, 300, 500 may be directly coupled to a delivery device (e.g., an endoscope, a catheter, etc.) that is positioned adjacent to a target treatment site within a subject (e.g., a patient), such as valve assembly 420 shown and described above.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description. The features described herein may be used alone or in combination and are not mutually exclusive.

What is claimed is:

1. A device for delivering an agent, comprising:
an inlet configured to receive a pressurized fluid;
an enclosure configured to store the agent; and
an actuator positioned between and in fluid communication with each of the inlet and the enclosure, the actuator including an opening;
wherein the actuator is configured to deliver the pressurized fluid from the inlet towards the enclosure in response to the opening being sealed, thereby inhibiting the pressurized fluid from exiting the device via the opening; and
wherein the actuator is configured to inhibit delivery of the pressurized fluid from the inlet towards the enclosure in response to the opening being unsealed, thereby allowing the pressurized fluid to exit the device via the opening.

2. The device of claim 1 further comprising:
a handle including the inlet and the actuator, wherein the actuator includes a button formed along the handle such that the opening is disposed along an exterior of the handle.

3. The device of claim 2, wherein the button is configured to receive a finger of a user to selectively seal and unseal the opening for delivering the pressurized fluid towards and away from the enclosure.

4. The device of claim 1, further comprising:
a bypass channel positioned between the actuator and the enclosure, the bypass channel being in fluid communication with the inlet via the actuator for receiving the pressurized fluid.

5. The device of claim 4, wherein the bypass channel is configured to receive a portion of the pressurized fluid from the actuator in response to the opening being sealed; and
wherein the bypass channel extends around the enclosure such that the bypass channel is configured to deliver the portion of the pressurized fluid received from the actuator to an outlet of the device that is positioned downstream from the enclosure.

6. The device of claim 5, further comprising:
a first valve disposed downstream from the bypass channel, wherein the first valve is configured to selectively direct the portion of the pressurized fluid towards the outlet based on a position of the first valve.

7. The device of claim 6, wherein when in a first position, the first valve is configured to close the bypass channel to inhibit the portion of the pressurized fluid from moving towards the outlet and exiting the device; and
wherein when in a second position, the first valve is configured to open the bypass channel to allow the portion of the pressurized fluid to move towards the outlet and exit the device.

8. The device of claim 6, further comprising:
a second valve disposed downstream from the enclosure, wherein the second valve is configured to selectively direct the agent stored in the enclosure towards the outlet based on a position of the second valve.

9. The device of claim 8, wherein when in a first position, the second valve is configured to close an opening of the enclosure to inhibit the agent from moving towards the outlet and exiting the device; and
wherein when in a second position, the second valve is configured to open the opening of the enclosure to allow the agent to move towards the outlet and exit the device.

10. The device of claim 1, wherein the enclosure includes an interior surface defining a cavity that is configured to store the agent, and an agitation mechanism extending from the interior surface into the cavity; and
wherein the agitation mechanism is configured to agitate the pressurized fluid received in the enclosure from the actuator and the agent stored in the cavity, thereby forming a mixture of the pressurized fluid and the agent within the enclosure for delivery.

11. The device of claim 10, wherein the agitation mechanism includes a plurality of ribs configured to abut against the agent in response to the pressurized fluid received in the enclosure moving the agent within the cavity.

12. The device of claim 10, wherein the agitation mechanism includes a rotatable disc that is configured to rotate within the enclosure and generate a vortex within the cavity in response to the pressurized fluid received in the enclosure moving the rotatable disc.

13. The device of claim 1, wherein the actuator includes a first actuator and the opening includes a first opening;
wherein the device further comprises:
an outlet; and
a second actuator positioned between and in fluid communication with each of the inlet and the outlet, wherein the second actuator includes a second opening.

14. The device of claim 13, wherein the second actuator is configured to deliver the pressurized fluid from the inlet towards the outlet in response to the second opening being sealed, thereby inhibiting the pressurized fluid from exiting the device via the second opening; and
wherein the second actuator is configured to inhibit delivery of the pressurized fluid from the inlet towards the outlet in response to the second opening being unsealed, thereby allowing the pressurized fluid to exit the device via the second opening.

15. The device of claim 14, wherein the first actuator is configured to selectively deliver a first portion of the pressurized fluid received from the inlet towards the enclosure and the second actuator is configured to selectively deliver a second portion of the pressurized fluid received from the inlet towards the outlet independently from one another based on each of the first opening and the second opening being sealed or unsealed.

16. A device for delivering an agent, comprising:
an enclosure configured to store the agent;
an inlet configured to receive a pressurized fluid;
an outlet configured to deliver the agent and the pressurized fluid out of the device;
a first actuator positioned between and fluidly coupled to the inlet and the enclosure, wherein the first actuator is configured to selectively guide the pressurized fluid from the inlet towards the enclosure for mixing with the agent when the first actuator is covered, and release the pressurized fluid from the inlet out of the device when the first actuator is uncovered; and
a second actuator positioned between and fluidly coupled to the inlet and the outlet, wherein the second actuator is configured to selectively guide the pressurized fluid from the inlet towards the outlet for when the second actuator is covered, and release the pressurized fluid from the inlet out of the device when the second actuator is uncovered.

17. The device of claim 16, wherein the first actuator and the second actuator are configured to simultaneously guide the pressurized fluid into the enclosure for mixing the agent and towards the outlet for delivering the agent released from the enclosure towards the outlet when each of the first actuator and the second actuator are covered.

18. The device of claim 16, wherein the first actuator and the second actuator are configured to guide the pressurized fluid away from the enclosure and towards the outlet for delivering the pressurized fluid towards the outlet in isolation from the agent when the first actuator is uncovered and the second actuator is covered.

19. The device of claim 16, wherein the first actuator and the second actuator are configured to guide the pressurized fluid away from the enclosure and the outlet, thereby releasing the pressurized fluid prior to mixing with the agent and entering the outlet, when each of the first actuator and the second actuator are uncovered.

20. A system for delivering an agent, comprising:
a first device including a handle and a delivery conduit that is in fluid communication with the handle; and
a second device including an actuator, an inlet configured to receive a pressurized fluid, and an enclosure configured to store the agent;
wherein the actuator is configured to fluidly couple the first device and the second device to selectively deliver the agent from the enclosure to the delivery conduit in response to the actuator moving from an unsealed state to a sealed state;
wherein the actuator is configured to divert the pressurized fluid received from the inlet away from the enclosure and out of the second device prior to the pressurized fluid entering the first device when in the unsealed state; and
wherein the actuator is configured to urge the pressurized fluid received from the inlet into the enclosure and out of the second device for receipt by the first device when in the sealed state.
